(19) United States
(12) Patent Application Publication
(10) Pub. No.: US 2008/0147099 A1
(43) Pub. Date: Jun. 19, 2008

(54) Bilayer patch device for hernia repair

(76) Inventor: Yih-Huei Uen, Yung Kang City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/638,480

(22) Filed: Dec. 14, 2006

(51) Int. Cl. A61B 17/08

(52) U.S. Cl. 606/151

(57) ABSTRACT

A bilayer patch device for hernia repair includes a first layer and a second layer. The first layer is cut to form locating flakes by cutting lines. The edges of the first layer and the second layer are connected to form a pocket. The second layer further comprises an auxiliary layer. The auxiliary layer further comprises a fin thereof. The patch is able to be flat attached to a cavity of the peritoneum for repairing a hernia.

### BACKGROUND OF THE INVENTION

1. Field of the Invention

The present invention relates to a bilayer patch device for hernia repair, in particular to a flat pocket-shaped patch which is able to be attached to a peritoneum cavity of the patient to increase the quality of hernia repair and to avoid recurrence of the hernia.

2. Description of the Prior Art

A conventional patch device for hernia repair, as described in the U.S. Pat. No. 6,241,768, uses a double-layer in a flat shape design, which made improvement with respect to a single-layer patch. However, this double-layer design can not be attached to the peritoneum cavity evenly because the patch can not be fixed to the cavity of the peritoneum. When the internal organs squirm, the patch moves along with the internal organs. That may cause the patch to change its position.

### SUMMARY OF THE INVENTION

It is the primary objective of the present invention to provide a bilayer patch device for hernia repair, which can be attached evenly to the cavity of the patient's peritoneum.

It is another objective of the present invention to provide a bilayer patch device for hernia repair, which is save in use.

It is a further objective of the present invention to provide a bilayer patch device for hernia repair, which is easy to use.

According to the present invention, there is provided a bilayer patch device for hernia repair comprising a first layer having cutting lines thereon to form locating flakes; and a second layer having its edge connected with the edge of said first layer to form a pocket therein.

In another embodiment of the present invention, there is provided a bilayer patch device for hernia repair comprising a first layer having cutting lines thereon to form locating flakes; and a second layer having its edge connected with the edge of said first layer to form a pocket therein, an auxiliary layer being provided on said second layer, by opening said locating flakes, said auxiliary layer being inserted through said first layer, wherein said auxiliary layer of said second layer comprises a fin.

### BRIEF DESCRIPTION OF THE DRAWINGS

FIG. 1 is an exploded view of a first embodiment of the present invention;

FIG. 2 is a perspective view of FIG. 1;

FIG. 3 is a side cross-sectional view of FIG. 1;

FIG. 4 is a cross-sectional view of the first embodiment of the present invention in an operating status; (Locating lines are inserted through locating flakes to be positioned to the cavity of the peritoneum);

FIG. 5 is a cross-sectional view of the first embodiment of the present invention in an operating status; (A tool is used to make the patch flat attached to the cavity.)

FIG. 6 is an exploded view of a second embodiment of the present invention;

FIG. 7 is a cross-sectional view of the second embodiment of the present invention;

FIG. 8 is an exploded view of a third embodiment of the present invention; and

FIG. 9 is a cross-sectional view of the third embodiment of the present invention.

### DETAILED DESCRIPTION OF THE PREFERRED EMBODIMENTS

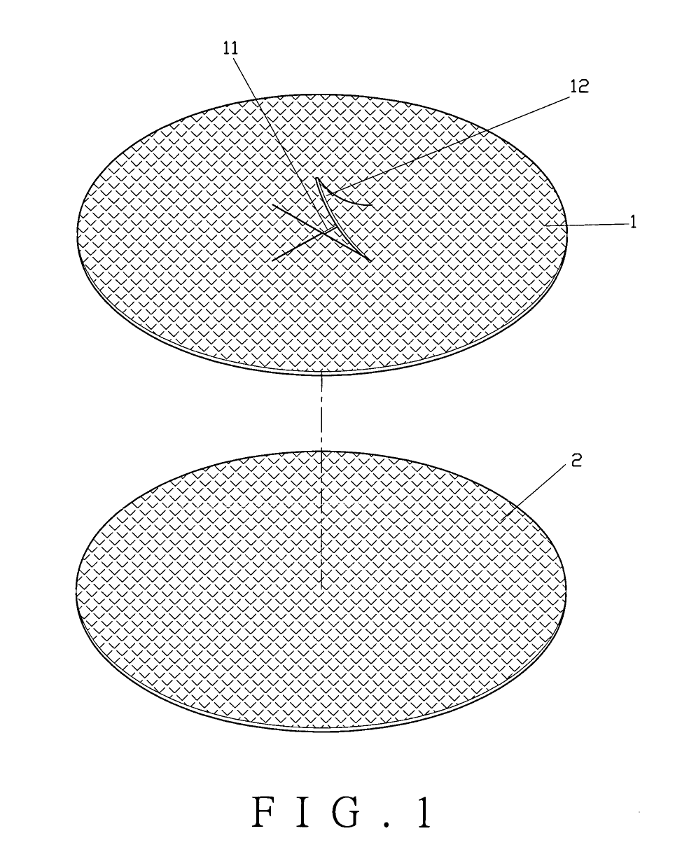

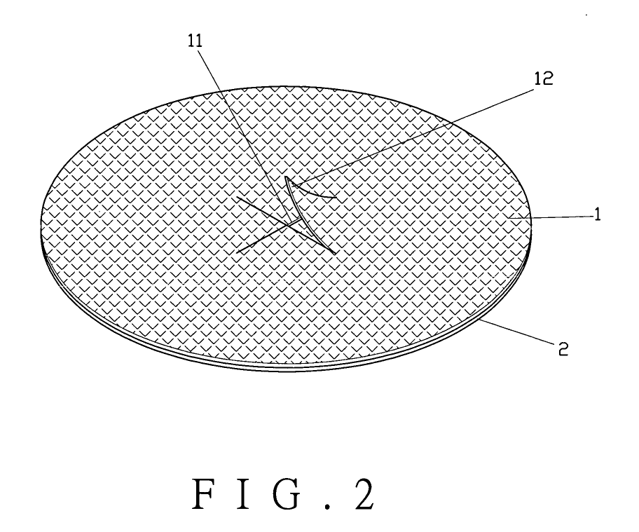

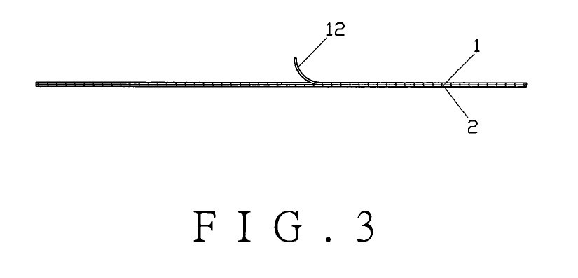

As shown in FIGS. 1 to 3, a bilayer patch device for hernia repair of a first embodiment of the present invention comprises a first layer 1 and a second layer 2.

The first layer 1 is cut to form locating flakes 12 by cutting lines 11. In this embodiment, the four cutting lines 11 are connected to a center point to form the four locating flakes 12. The four locating flakes 12 are opened to form an opening. This embodiment may use two, three or five cutting lines 11. All of the cutting lines 11 are connected at a common point.

The second layer 2 has its edge connected with the edge of the first layer 1 to form a pocket. The connecting edges of the first layer 1 and the second layer 2 are connected with threads, glue or using high wave frequency method.

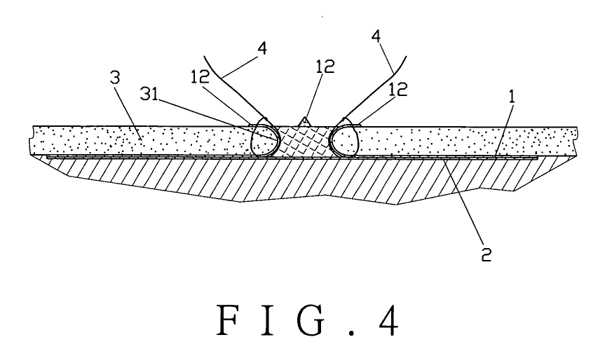

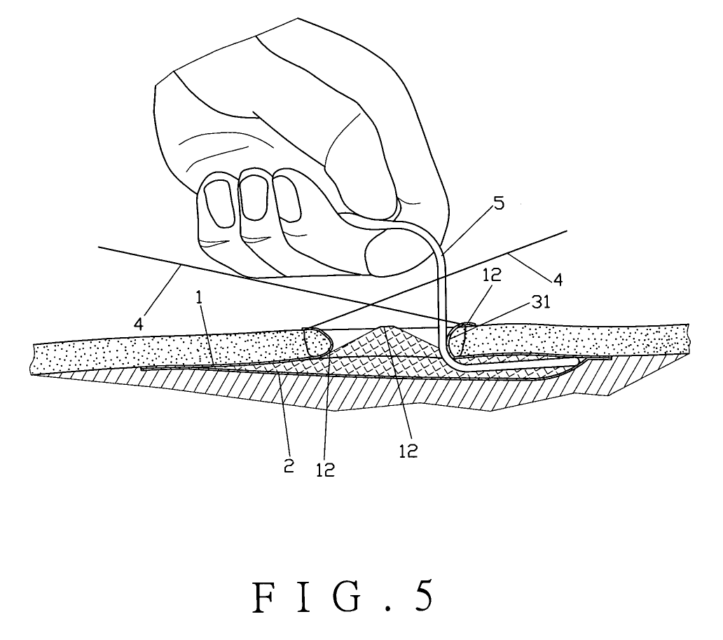

In practice the first embodiment of the present invention, as shown in FIGS. 4 and 5, the bilayer patch device of the present invention is placed into a cavity 31 of the patient's peritoneum 3 with locating threads 4 inserted through the locating flakes 12. The surgeon inserts a finger into the pocket formed by the first layer 1 and the second layer 2 to place the bilayer patch device into the peritoneum 3. The surgeon may use a tool 5 to flat the bilayer patch device, as shown in FIG. 5, before it is pulled out. The bilayer patch device is flat attached to the peritoneum 3 while the locating flakes 12 keep the bilayer patch device in place.

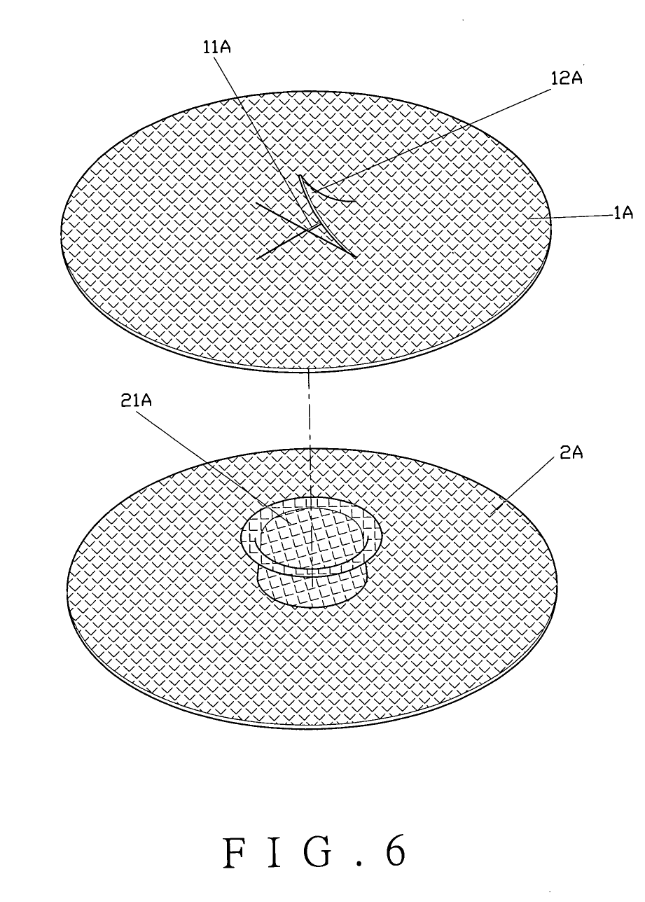

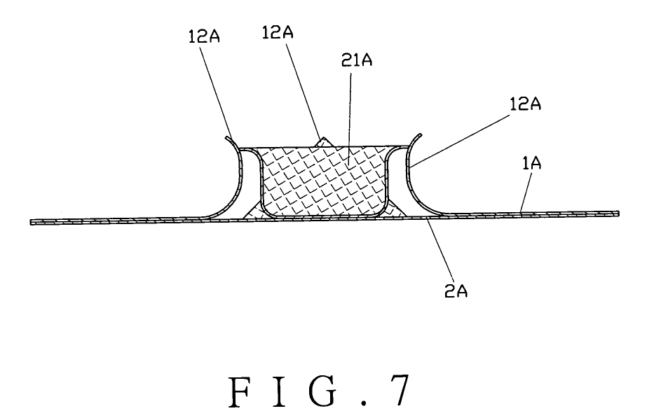

As shown in FIGS. 6 and 7, a second embodiment of the present invention comprises a first layer 1A having cutting lines 11A thereon to form locating flakes 12A. A second layer 2A has its edge connected with the edge of the first layer 1A to form a pocket. The second layer 2A further comprises an auxiliary layer 21A on the top of the second layer 2A to be inserted through the locating flakes 12A of the first layer 1A. The auxiliary layer 21A is in a shallow barrel. An open end is formed between the auxiliary layer 21A and the locating flakes 12A.

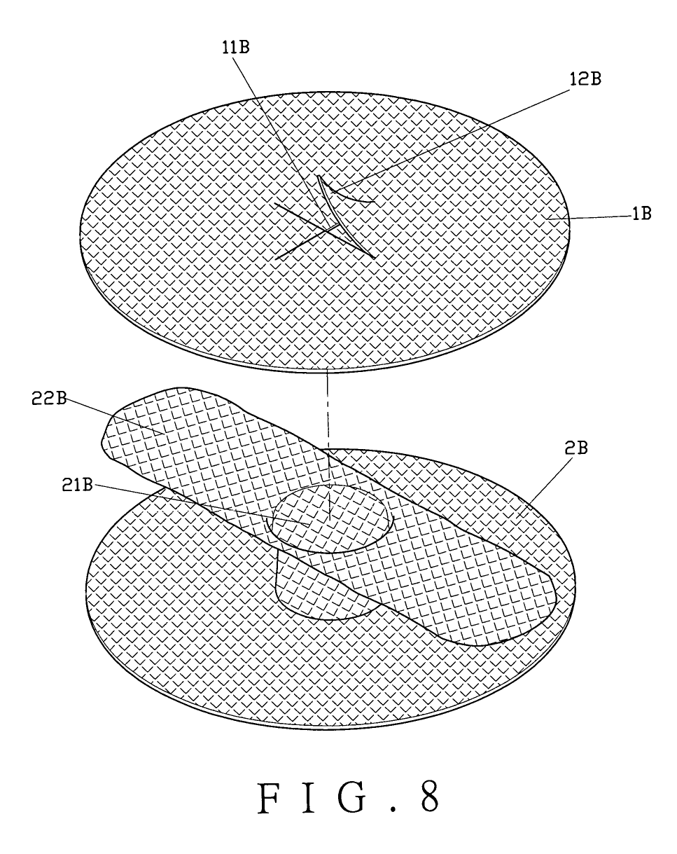

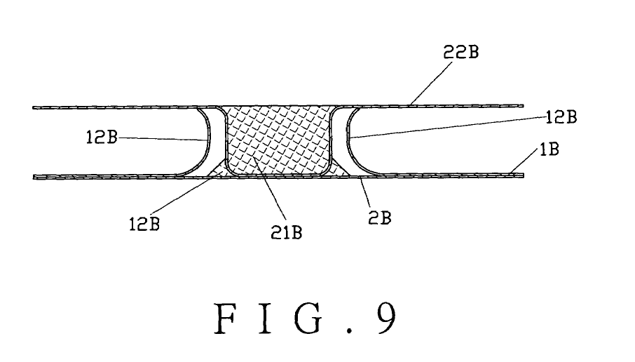

As shown in FIGS. 8 and 9, a third embodiment of the present invention comprises a first layer 1B having cutting lines 11B thereon to form locating flakes 12B. A second layer 2B has its edge connected with the edge of the first layer 1B to form a pocket. The second layer 2A further comprises an auxiliary layer 21B on the top thereof. The auxiliary layer 21B comprises a fin 22B to be inserted through the locating flakes 12B of the first layer 1B. The auxiliary layer 21B is in a shallow barrel. The fin 22B facilitates the user's operation. An open end is formed between the auxiliary layer 21B and the locating flakes 12B.

What is claimed is:

1. A bilayer patch device for hernia repair comprising:
a first layer having cutting lines thereon to form locating flakes; and
a second layer having its edge connected with the edge of said first layer to form a pocket therein.

2. A bilayer patch device for hernia repair comprising:
a first layer having cutting lines thereon to form locating flakes; and
a second layer having its edge connected with the edge of said first layer to form a pocket therein, an auxiliary layer being provided on said second layer, by opening said locating flakes, said auxiliary layer being inserted through said first layer.

3. The bilayer patch device for hernia repair, as recited in claim 2, wherein said auxiliary layer of said second layer comprises a fin.
